(19) United States
(12) Patent Application Publication
(10) Pub. No.: US 2012/0021030 A1
(43) Pub. Date: Jan. 26, 2012

(54) Liquid Composition Containing Fusiform Particles for Cosmetic Use

(76) Inventors: Shinichi Matsufuji, Kanagawa (JP); Makoto Kawamoto, Kanagawa (JP)

(21) Appl. No.: 13/260,451

(22) PCT Filed: Mar. 27, 2009
(86) PCT No.: PCT/JP2009/001421

§ 371 Date: Sep. 26, 2011

(51) Int. Cl. A61K 8/02; A61Q 1/02; A61Q 17/04; A61Q 1/00

(52) U.S. Cl. 424/401

(57) ABSTRACT

A liquid composition for cosmetic use containing 5 to 50 wt % of fusiform particles in a cosmetically acceptable medium, the fusiform particles being made of an organosilicone having a crosslinked polysiloxane structure, and having diameters along major axis L₁ between approximately 0.05 micro meter and approximately 20 micro meter, diameters along minor axes L₂ between approximately 0.03 micro meter and approximately 15 micro meter, and L₁/L₂ between approximately 1.1 and approximately 3.3.

### TECHNICAL FIELD

The present invention relates to a liquid composition, in particular liquid foundation, for cosmetic use which contains particles having a non (hemi)spherical shape such as a fusiform shape, also named a “rugby ball” shape according to the invention.

By “liquid” composition according to the invention, we mean liquid or fluid composition, by opposition to solid composition whose high hardness does not permit the composition to flow under its own weight.

1. Background

It is desired to provide a liquid composition, in particular a liquid foundation, with an ability to preserve moisture after applied to the skin. However, other features such as a good transparency and less powdery appearance, for a natural make-up result in particular for a foundation product, are also searched for.

2. Disclosure of Invention

In view of the fact that the products now available still require improved moisture preservability yet having simultaneously a good transparency and less powdery appearance after the passage of time, the inventors of the present invention have tested numerous ingredients to be included in a liquid foundation and discovered that inclusion of fusiform particles can provide liquid foundations with desired features and thereby completed the present invention.

According to an aspect of the present invention, the inventors propose a liquid composition for cosmetic use containing 3 to 60 wt %, preferaly 5 to 50 wt % of fusiform particles in a cosmetically acceptable medium, the fusiform particles being made of an organosilicone having a crosslinked polysiloxane structure, and having diameters along major axis L₁ between approximately 0.05 micro meter and approximately 20 micro meter, diameters along minor axes L₂ between approximately 0.03 micro meter and approximately 15 micro meter, and L₁/L₂ between approximately 1.1 and approximately 3.3.

The fusiform particles as well as the methods for obtaining the fusiform particles are described, for example, in the Japanese Patent Application, Publication No. 2003-171465 filed by Takemoto Fat and Oil.

In the present specification, unless otherwise specified, the term “cosmetic use” includes uses for so-called cosmetic and dermatological purposes.

Another subject-matter of the invention is a cosmetic process for making up or caring for human keratinous substances, such as the skin, hair or nails, and more particularly the skin.

The composition according to the invention can be a composition for making up or caring for the skin and can be provided in the form of a product for making up or caring for the face, a product for making up or caring for the body or an antisun product, in particular in the form of an aqueous solution, a gel or an emulsion. In a preferred embodiment, the composition is a foundation in the form of an aqueous solution, a gel or an emulsion.

A fusiform or “rugby ball” shape in this specification refers to such a shape that a sphere is extended in one direction so that there is a major axis along which the fusiform particle has the largest diameter L₁ which is between approximately 0.05 micro meter and approximately 20 micro meter and two minor axes L₂ perpendicular to the major axis and to each other along which the fusiform particle has the smallest diameter which is between approximately 0.03 micro meter and approximately 15 micro meter, and L₁/L₂ between approximately 1.1 and approximately 3.3.

The organosilicone material having a crosslinked polysiloxane structure preferably comprises, indeed even is composed of, units of formula (I): SiO₂, and of formula (II): R¹SiO_{1.5},

in which R¹ denotes an organic group having a carbon atom directly connected to the silicon atom. The organic group can be a reactive organic group or an unreactive organic group and preferably an unreactive organic group.

The organosilicone material having a crosslinked polysiloxane structure also preferably comprises first, second and third siloxane units which are formula (I): SiO₂, formula (II): R¹SiO_{1.5} and formula (III): R²R³SiO, respectively, wherein R¹, R² and R³ are any one of organic groups, same or different, having a carbon atom directly linked to a silicon atom.

R² and R³ may be either a non-reactive organic group or an organic group having no reactive group or a reactive organic group or an organic group having a reactive group, independently. However, it is preferred that at least either one of R² and R³ a reactive organic group or an organic group having a reactive group.

The unreactive organic group can be a C₁-C₄ alkyl group, in particular a methyl, ethyl, propyl or butyl group, or a phenyl group and preferably a methyl group.

The reactive organic group can be an epoxy group, a (meth)acryloyloxy group, an alkenyl group, a mercaptoalkyl, aminoalkyl or haloalkyl group, a glyceroxy group, a ureido group or a cyano group. Preferably, the reactive organic group can be an epoxy group, a (meth)acryloyloxy group, an alkenyl group or a mercaptoalkyl or aminoalkyl group. The reactive organic group generally comprises from 2 to 6 carbon atoms, in particular from 2 to 4 carbon atoms.

Mention may be made, as epoxy group, of a 2-glycidoxyethyl group, a 3-glycidoxypropyl group or a 2-(3,4-epoxycyclohexyl)propyl group.

Mention may be made, as (meth)acryloyloxy group, of a 3-methacryloyloxypropyl group or a 3-acryloyloxypropyl group.

Mention may be made, as alkenyl group, of a vinyl, allyl or isopropenyl group.

Mention may be made, as mercaptoalkyl group, of a mercaptopropyl or mercaptoethyl group.

Mention may be made, as aminoalkyl group, of a 3-[(2-aminoethyl)amino]propyl group, a 3-aminopropyl group or an N,N-dimethylaminopropyl group.

Mention may be made, as haloalkyl group, of a 3-chloropropyl group or a trifluoropropyl group.

Mention may be made, as glyceroxy group, of a 3-glyceroxypropyl group or a 2-glyceroxyethyl group.

Mention may be made, as ureido group, of a 2-ureidoethyl group.

Mention may be made, as cyano group, of a cyanopropyl or cyanoethyl group.

Preferably, in the unit of formula (II), R¹ denotes a methyl group.

Advantageously, the organosilicone material comprises the units (I) and (II) according to a unit (I)/unit (II) molar ratio ranging from 30/70 to 50/50, preferably ranging from 35/65 to 45/55.

Also advantageously, the organosilicone material comprises the units (I), (II) and (III) so that the molar ratio of the molar sum of the first siloxane unit (I) and the second unit (II) over the third siloxane unit (III) is between 99:1 to 50:50, more preferably between 90:10 to 60:40, approximately. The molar ratio of the first siloxane unit (I) with respect to the second siloxane unit (II) may preferably be between 23:77 and 40:60, approximately.

The organosilicone particles can in particular be capable of being obtained according to a process comprising:

(a) the introduction into an aqueous medium, in the presence of at least one hydrolysis catalyst and optionally of at least one surfactant, of a compound (IV) of formula SiX₄ and of a compound (V) of formula R⁴SiY₃, where X and Y denote, independently of one another, a C₁-C₄ alkoxy group, an alkoxyethoxy group including a C₁-C₄ alkoxy group, a C₂-C₄ acyloxy group, an N,N-dialkylamino group including a C₁-C₄ alkyl group, a hydroxyl group, a halogen atom or a hydrogen atom and R denotes an organic group comprising a carbon atom connected directly to the silicon atom; and

(b) the operation in which the mixture resulting from stage (a) is brought into contact with an aqueous solution including at least one polymerization catalyst and optionally at least one surfactant, at a temperature of between 30 and 85 degrees Celsius, for at least two hours.

Stage (a) corresponds to a hydrolysis reaction and stage (b) corresponds to a condensation reaction.

In stage (a), the molar ratio of the compound (IV) to the compound (V) generally ranges from 30/70 to 50/50, advantageously from 35/65 to 45/45, and is preferentially 40/60. The ratio by weight of the water to the total of the compounds (IV) and (V) preferably ranges from 10/90 to 70/30. The order of introduction of the compounds (IV) and (V) generally depends on their rate of hydrolysis. The temperature of the hydrolysis reaction generally ranges from 0 to 40 degrees Celsius and usually does not exceed 30 degrees Celsius in order to prevent premature condensation of the compounds.23:77 to 40:60

The organosilicone particles can also be capable of being obtained according to a process comprising:

(a) the introduction into an aqueous medium, in the presence of at least one hydrolysis catalyst, of a silanol group forming silicon compound (IV) of formula SiX₄, of a silanol group forming silicon compound (V) of formula R⁴SiY₃, and of a silanol group forming silicon compound (VI) of formula R⁵R⁶SiZ₂, at the molar ratio for (the silanol group forming silicon compound (IV) and the silanol group forming silicon compound (V): the silanol group forming silicon compound (VI) to be 99:1 to 50:50, preferably 90:10 to 60:40 so that the the silanol group forming silicon compounds are hydrolyzed to generate a silanol compound (it is furthermore preferred that the silanol forming silicon compounds of formula 4 and 5 are at the molar ratio of 23:77 to 40:60); and

(b) causing condensing reaction of the generated silanol compound in aqueous medium in the presence of at least one hydrolysis catalyst

For the X, Y and Z groups of the compounds (IV), (V) and (VI):

mention may be made, as C₁-C₄ alkoxy group, of the methoxy or ethoxy groups;

mention may be made, as alkoxyethoxy group including a C₁-C₄ alkoxy group, of the methoxyethoxy or butoxyethoxy groups;

mention may be made, as C₂-C₄ acyloxy group, of the acetoxy or propionyloxy groups;

mention may be made, as N,N-dialkylamino group including a C₁-C₄ alkyl group, of the dimethylamino or diethylamino groups;

mention may be made, as halogen atom, of the chlorine or bromine atoms.

Mention may be made, as compounds of formula (IV), of tetramethoxysilane, tetraethoxysilane, tetrabutoxysilane, trimethoxyethoxysilane, tributoxy-ethoxysilane, tetraacetoxysilane, tetrapropioxysilane, tetra(dimethylamino)silane, tetra(diethylamino)silane, silanetetraol, chlorosilanetriol, dichlorodisilanol, tetrachlorosilane or chlorotrihydrosilane. Preferably, the compound of formula (IV) is chosen from tetra-methoxysilane, tetraethoxysilane, tetrabutoxysilane and their mixtures.

The compound of formula (IV) results, after the polymerization reaction, in the formation of the units of formula (I).

The compound of formula (V) results, after the polymerization reaction, in the formation of the units of formula (II).

The R group in the compound of formula (V) has the meaning as described for the R¹ group for the compound of formula (II).

Mention may be made, as examples of compounds of formula (V) comprising an unreactive organic group R, of methyltrimethoxysilane, ethyltriethoxysilane, propyltributoxysilane, butyltributoxysilane, phenyltri-methoxyethoxysilane, methyltributoxyethoxysilane, methyltriacetoxysilane, methyltripropioxysilane, methyltri(dimethylamino)silane, methyltri(diethyl-amino)silane, methylsilanetriol, methylchlorodisilanol, methyltrichlorosilane or methyltrihydrosilane.

Mention may be made, as examples of compounds of formula (V) comprising a reactive organic group R, of:
silanes having an epoxy group, such as (3-glycidoxy-propyl)trimethoxysilane,

(3-glycidoxypropyl)triethoxy-silane, [2-(3,4-epoxycyclohexyl)ethyl]trimethoxy-silane,

(3-glycidoxypropyl)methyldimethoxysilane,

(2-glycidoxy-ethyl)methyldimethoxysilane,

(3-glycidoxypropyl)di-methylmethoxysilane or

(2-glycidoxyethyl)dimethyl-methoxysilane;
silanes having a (meth)acryloyloxy group, such as

(3-methacryloyloxypropyl)trimethoxysilane or

(3-acryl-oyloxypropyl)trimethoxysilane;
silanes having an alkenyl group, such as vinyltri-methoxysilane, allyltrimethoxysilane or isopropenyltri-methoxysilane;
silanes having a mercapto group, such as mercapto-propyltrimethoxysilane or mercaptoethyltrimethoxy-silane;
silanes having an aminoalkyl group, such as (3-amino-propyl)trimethoxysilane,

(3-[(2-aminoethyl)amino]-propyl)trimethoxysilane,

(N,N-dimethylaminopropyl)tri-methoxysilane or

(N,N-dimethylaminoethyl)trimethoxy-silane;
silanes having a haloalkyl group, such as (3-chloro-propyl)trimethoxysilane or trifluoropropyltrimethoxy-silane;
silanes having a glyceroxy group, such as (3-glyceroxy-propyl)trimethoxysilane or di(3-glyceroxypropyl)di-methoxysilane;
silanes having a ureido group, such as (3-ureido-propyl)trimethoxysilane,

(3-ureidopropyl)methyldi-methoxysilane or (3-ureidopropyl)dimethylmethoxy-silane;
silanes having a cyano group, such as cyanopropyltri-methoxysilane, cyanopropyl-methyldimethoxysilane or cyanopropyldimethylmethoxysilane.

Preferably, the compound of formula (V) comprising a reactive organic group R is chosen from silanes having an epoxy group, silanes having a (meth)acryloyloxy group, silanes having an alkenyl group, silanes having a mercapto group or silanes having an aminoalkyl group.

Examples of compounds (IV) and (V) which are preferred for the implementation of this invention are respectively tetraethoxysilane and methyltri-methoxysilane.

Use may independently be made, as hydrolysis and polymerization catalysts, of basic catalysts, such as sodium hydroxide, potassium hydroxide, sodium carbonate, sodium hydrogencarbonate or amines (such as ammonia, trimethylamine, triethylamine or tetramethyl-ammonium hydroxide), or acidic catalysts chosen from organic acids, such as citric acid, acetic acid, methanesulphonic acid, p-toluenesulphonic acid, dodecyl-benzenesulphonic acid or dodecylsulphonic acid, or inorganic acids, such as hydrochloric acid, sulphuric acid or phosphoric acid. When it is present, the surfactant used is preferably a nonionic or anionic surfactant or a mixture of the two. Sodium dodecyl-benzenesulphonate can be used as anionic surfactant. The end of the hydrolysis is marked by the disappearance of the products (IV) and (V), which are insoluble in water, and the production of a homogeneous liquid layer.

The silanol forming silicon compounds of formula (VI) forms siloxane unit (III) as a result. The group Z in formula (VI) may be selected from the group listed for group X in formula (IV). R⁵ and R⁶ in formula (VI) may be selected from the group described for R² and R³ in formula (III).

The condensation stage (b) can use the same catalyst as the hydrolysis stage or another catalyst chosen from those mentioned above.

On conclusion of this process, a suspension in water of fine organosilicone particles is obtained, which particles can optionally be separated subsequently from their medium. The process described above can thus comprise an additional stage of filtration, for example on a membrane filter, of the product resulting from stage (b), optionally followed by a stage of centrifuging the filtrate, intended to separate the particles from the liquid medium, and then by a stage of drying the particles. Other separation methods can, of course, be employed.

In a particular embodiment, the fusiform particles have slits along their major axes.

According to the present invention, the fusiform particles may preferably be between approximately 5 wt % and approximately 50 wt %, preferably approximately 10 wt % and approximately 30 wt %, more preferably between approximately 10 wt % and approximately 20 wt % of the liquid composition. The crosslinked polysiloxane may comprise first, second and third siloxane units which are SiO₂, R¹SiO_{1.5} and R²R³SiO, respectively, wherein R¹, R² and R³ are any one of organic groups, same or different, having a carbon atom directly linked to a silicon atom.

The molar ratio of the third siloxane unit with respect to the molar sum of the first, second and third siloxane units is between 1 to 50 percent, approximately.

The molar ratio of the molar sum of the first siloxane unit and the second unit over the third siloxane unit may preferably between 99:1 to 50:50, more preferably between 90:10 to 60:40, approximately. The molar ratio of the first siloxane unit with respect to the second unit may preferably be between 23:77 and 40:60, approximately.

The molar ratio of the first siloxane unit with respect to the second siloxane unit is between 23:77 and 40:60, approximately. In the liquid composition according to the present invention, R¹ and at least one of R² and R³ may preferably selected from the group consisting of epoxy group, (meta) acryloxy group, mercaptoalkyl group, aminoalkyl group and organic groups having any one of preceding groups.

The above-described R¹ and at least one of R² and R³ may be selected from the group consisting of epoxy group, (meta) acryloxy group, mercaptoalkyl group, aminoalkyl group and organic groups having any one of preceding groups.

In particular, the fusiform particles have slits along their major axes.

The liquid composition according to the present invention may further comprise at least one compound chosen from water, hydrophilic solvents, lipophilic solvents, oils, waxes and pasty fatty substances, and mixtures thereof. The oils may be chosen from volatile or non-volatile hydrocarbon-based oils of animal, plant or mineral origin, synthetic oils, silicone oils and fluoro oils, and mixtures thereof. The liquid composition according to the present invention may be in the form of an aqueous solution, a lotion, an oil gel, an oil-in-water or water-in-oil simple emulsion or a multiple emulsion.

The liquid composition according to the present invention may further comprise a filler, a pigment and another cosmetically acceptable ingredient.

The filler may be selected from the group consisting of talc, mica, silica, kaolin, powder formed of polyamide, of poly - - - alanine and of polyethylene, powders formed of tetrafluoro-ethylene polymers, lauryllysine, starch, boron nitride, polymeric hollow microspheres of poly(vinylidene chloride)/acrylonitrile or of acrylic acid copolymers, silicone resin microbeads, particles formed of polyorgano-siloxane elastomers, precipitated calcium carbonate, magnesium carbonate, basic magnesium carbonate, hydroxyapatite, barium sulphate, aluminium oxides, polyurethane powders, composite fillers, hollow silica microspheres, glass or ceramic microcapsules, or metal soaps derived from organic carboxylic acids having from 8 to 22 carbon atoms, preferably from 12 to 18 carbon atoms, for example zinc stearate, magnesium stearate, lithium stearate, zinc laurate and magnesium myristate

Other fillers are shown by Table 1 below.

| | | | Morphology | | |
|---|---|---|---|---|---|
| | Surface | Chemistry | | Mean | |
| Name | treatment | (composition) | Shape | Size (μm) | Size Span |
| Amiboae LI | NT | Lauroyl Lysine | lamellar | 11.7 | 1.4 |
| Mica Concord 1000 | NT | Mica | lamellar | 8.3 | 2.7 |
| SERICITE S [symbol] 152 | NT | Mica | lamellar | 7.8 | 2.3 |
| BC | | | | | |
| Ser [symbol] cite S [symbol] | NT | Mica | lamellar | 7.3 | 2.2 |
| MEARLM:CA CF | NT | Mica | lamellar | 18.5 | 1.9 |
| MM/CF | | | | | |
| [symbol] 88 BC | NT | Talc | lamellar | 10.4 | 3.1 |
| Cer [symbol] st 9205 [symbol] | NT | [symbol] | flake | 3.5 | 0.9 |
| PUHP 3008 | NT | Boron Nitride | flake | 5.4 | 1.8 |
| SO [symbol] OJCH BN | NT | Boron Nitride | lamellar | 9.0 | 1.7 |
| POWDER CC6058 | | | | | |
| Cost [symbol] C [symbol] | NT | Ka [symbol] | flake | 1.6 | 1.7 |
| TALC P3 | NT | Talc | lamellar | 4.4 | 1.7 |
| Biron LF 200C | NT | Oxycalorice Biam [symbol] | flake | 7.5 | 1.5 |
| LLD-5 BaSO4 | LL-5% | Barium Sulfate | flake | 4.3 | 1.6 |
| Coverleaf AR8C | NT | SiO2—BO2—Al2O3 on | flake | 4.3 | 1.0 |
| | | Talc | | | |
| Boron Nitrate SHP 3 | NT | Boron Nitride | flake | 6.7 | 1.3 |
| MICA POWDER | NT | Mica | lamellar | 11.4 | 1.9 |
| Y-2300 | | | | | |
| Optimat 1430 OR | NT | Perlite | lamellar | 16.4 | 1.9 |
| Pea [symbol] 2600LVS | NT | Oxycalorice Bismum | flake | 5.5 | 1.9 |
| Luzenac 00 | NT | Talc | lamellar | 14.2 | 2.4 |
| Luzenac Pharma M | NT | Talc | lamellar | 7.6 | 1.7 |
| Flake-shaped | NT | Barium sulfate | flake | 5.1 | 2.2 |
| Barium Sulfate HP | | | | | |
| Coverleaf AR100 | NT | S•O2—Al2O3 on Talc | flake | 3.5 | 1.1 |
| MTD010FYX | NT | Glass | lamellar | 13.4 | 2.6 |
| (6001) | | | | | |
| MTD025FYX | NT | Glass | lamellar | 20.9 | 2.2 |
| (6002) | | | | | |
| MTD010FYX | Si-2% | Glass | lamellar | 19.0 | 2.3 |
| (6009) | | | | | |
| MTD025FYX | Si-2% | Glass | lamellar | 38.6 | 1.9 |
| (6010) | | | | | |
| NL T30-2WA | NT | TiO2 or Silica | flake | 4.5 | 1.3 |
| Shadeleaf A | NT | ZnO or Mica-Barium | flake | 5.6 | 1.4 |
| | | sulfate | | | |
| B [symbol] sealer | NT | TiO2 or Mica | flake | 5.6 | 1.3 |
| Boroneige 1201 | NT | Boron Nitride | lamellar | 9.0 | 1.6 |
| Boroneige | NT | Boron Nitride | lamellar | 2.7 | 2.2 |
| Superfine | | | | | |
| Boroneige 501 | NT | Boron Nitride | lamellar | 7.2 | 1.9 |
| TBN 02 | NT | Boron Nitride | lamellar | 2.5 | 1.5 |
| Naturaleaf powder | NT | TiO2 or Mica | flake | 6.0 | 1.3 |
| | | Barium sulfate | | | |
| PDM 20L | NT | Synthetic Mica | lamellar | 22.7 | 1.6 |
| PDM 5L | NT | Synthetic Mica | lamellar | 7.2 | 1.5 |
| Se [symbol] te FSE | NT | Mica | lamellar | 7.6 | 1.8 |
| JA-4BR | NT | Talc | lamellar | 8.0 | 2.0 |
| Flake shaped | NT | Barium sulfate | flake | 4.1 | 1.9 |
| Barium Sulfate H | | | | | |

NT: no treatment
LL-5% = treated with 5% Lauroyl lysine [symbol]
indicates data missing or illegible when filed

The composition according to the invention may also contain pigments. The term pigments should be understood to mean particles of any shape, white or colored, inorganic or organic, which are insoluble in the physiological medium and which are intended to color the composition. The pigments can be white and/or colored and inorganic and/or organic.

According to a specific embodiment, the composition according to the invention can comprise at least one pigment chosen from inorganic pigments. These inorganic pigments can in particular be chosen from metal oxide pigments.

Mention may be made, among inorganic pigments, of titanium dioxide, optionally surface treated, zirconium or cerium oxides, and also zinc, iron (black, yellow or red) or chromium oxides, manganese violet, ultramarine blue, chromium hydrate and ferric blue, and metal powders, such as aluminium powder or copper powder, and their mixtures.

According to a preferred embodiment, the inorganic pigments, in particular metal oxide pigments, present in the composition according to the invention are chosen from titanium dioxide, zinc oxide and/or iron oxide.

According to a specific embodiment, the composition may comprise at least two different pigments. According to an embodiment which is also preferred, the composition may comprise at least one first pigment chosen from iron oxides and at least one second titanium dioxide pigment.

In addition to inorganic pigments, the composition according to the invention can comprise organic pigments. Mention may be made, among organic pigments, of carbon black, pigments of D & C type and lakes based on cochineal carmine, barium, strontium, calcium or aluminium.

The pigment can also comprise goniochromatic pigments. These pigments exhibit a relatively large change in color with the angle of observation. The goniochromatic pigment can be chosen, for example, from pigments comprising a multilayer interference structure and liquid crystal pigments.

In the case of a multilayer structure, the latter can comprise, for example, at least two layers, each layer, independently or not of the other layer(s), being produced, for example, from at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and their combinations.

The goniochromatic agents comprising multilayer structures are in particular those described in the following documents: U.S. Pat. No. 3,438,796, EP-A-227,423, U.S. Pat. No. 5,135,812, EP-A-170,439, EP-A-341,002, U.S. Pat. No. 4,930,866, U.S. Pat. No. 5,641,719, EP-A-472,371, EP-A-395,410, EP-A-753,545, EP-A-768,343, EP-A-571,836, EP-A-708,154, EP-A-579,091, U.S. Pat. No. 5,411,586, U.S. Pat. No. 5,364,467, WO-A-97/39066, DE-A-4,225,031, WO-9517479 (BASF) and DE-A-196,14637. They are provided in the form of flakes with a metallic color.

The multilayer structures which can be used in the invention are, for example, the following structures: Al/SiO₂/Al/SiO₂/Al; Cr/MgF₂/Al/MgF₂/Al; MoS₂/SiO₂/Al/SiO₂/MoS₂; Fe₂O₃/SiO₂/Al/SiO₂/Fe₂O₃; Fe₂O₃/SiO₂/Fe₂O₃/SiO₂/Fe₂O₃; MoS₂/SiO₂/mica oxide/SiO₂/MoS₂; Fe₂O₃/SiO₂/mica oxide/SiO₂/Fe₂O₃. Different colors are obtained depending on the thicknesses of the different layers. Thus, with the structure Fe₂O₃/SiO₂/Al/SiO₂/Fe₂O₃, the color changes from golden-green to grey-red for SiO₂ layers with a thickness of 320 to 350 nm; from red to golden for SiO₂ layers with a thickness of 380 to 400 nm; from purple to green for SiO₂ layers with a thickness of 410 to 420 nm; and from copper to red for SiO₂ layers with a thickness of 430 to 440 nm.

Consequently, the multilayer structure can be essentially inorganic or organic. Different colors are obtained depending on the thickness of each of the different layers. The goniochromatic pigments comprising a multilayer interference structure according to the invention are in particular those described in the following documents: U.S. Pat. No. 3,438,796, EP-A-227,423, U.S. Pat. No. 5,135,812, EP-A-170,439, EP-A-341,002, U.S. Pat. No. 4,930,866, U.S. Pat. No. 5,641,719, EP-A-472,371, EP-A-395,410, EP-A-753,545, EP-A-768,343, EP-A-571,836, EP-A-708,154, EP-A-579,091, U.S. Pat. No. 5,411,586, U.S. Pat. No. 5,364,467, WO-A-97/39066, DE-A-4,225,031, WO-9517479 (BASF) and DE-A-196,14,637, and their combinations. They are provided in the form of flakes with a metallic color.

The goniochromatic pigment comprising a multilayer interference structure can be chosen from the group consisting of the following commercial goniochromatic pigments: Infinite Colors from Shiseido, Sicopearl Fantastico from BASF, Colorstream, Xirallic and Xirona from Merck, Colorglitter from Flex, and their mixtures.

Liquid crystal pigments are described in particular in Application EP-A-1,046,692.

Use may in particular be made, as liquid crystal particles, of those known under the CTFA name Polyacrylate-4 and sold under the names “Helicone™ HC Sapphire”, “Helicone™ HC Scarabeus”, “Helicone™ HC Jade”, “Helicone™ HC Maple”, “Helicone™ HC XL Sapphire”, “Helicone™ HC XL Scarabeus”, “Helicone™ HC XL Jade” and “Helicone™ HC XL Maple” by Wacker.

The pigments can be present in the composition according to the invention in a content ranging from 1 to 30% by weight, with respect to the total weight of the composition, preferably from 5 to 20% by weight and more preferably from 5 to 15% by weight.

Another cosmetically acceptable ingredient is selected from the group consisting of waxes, preservatives, cosmetic active principles, moisturizing agents, UV screening agents, thickeners, water, surfactants, binders and fragrances.

The above-described surfactant may be one or more of zinc stearate, the binder includes a mineral oil and phenyl trimethicone. The preservative may be phenoxyethanol.

The liquid composition according to the present invention may be one of foundation, make-up, skin care, sun care, in particular in the form of an aqueous solution, a lotion, an oil gel, an oil-in-water or water-in-oil simple emulsion or a multiple emulsion.

Cosmetically acceptable other ingredients are, for example, waxes, preservatives, cosmetic active principles, moisturizing agents, UV screening agents, thickeners, water, surfactants, binders or fragrances. The cosmetically acceptable other ingredients may be one or more of pulverulent phases including spherical solid particles, boron nitride particles, lamellar particles, additional particles, fatty binders, waxes, pasty fatty substances, and other additives.

Fatty binder

The composition according to the invention may comprise at least one fatty binder.

The term fatty binder is understood to mean, within the meaning of the present application, a fatty phase which generally comprises at least one oil. This type of fatty phase is used in particular as dispersing medium for the pulverulent phase. Advantageously, the fatty binder can comprise at least one oil.

The oil can be chosen from the oils conventionally used as binder in loose or compact powders. These oils can in particular be chosen from:

mink oil, turtle oil, soybean oil, grape seed oil, sesame oil, corn oil, rapeseed oil, sunflower oil, cottonseed oil, avocado oil, olive oil, castor oil, jojoba oil or peanut oil;

hydrocarbon oils, such as liquid paraffins, squalane or liquid petrolatum;

fatty esters, such as isopropyl myristate, isopropyl palmitate, butyl stearate, isodecyl stearate, isocetyl stearate, hexyl laurate, isononyl isononanoate, 2-ethylhexyl palmitate, 2-hexyldecyl laurate, 2-octyldecyl palmitate, 2-octyldodecyl myristate, 2-octyldodecyl lactate, di(2-ethylhexyl) succinate, diisostearyl malate, glyceryl triiso-stearate or diglyceryl triisostearate;

silicone oils, such as polymethylsiloxanes, polymethylphenylsiloxanes, polysiloxanes modified by fatty acids, fatty alcohols or polyoxyalkylenes, fluorosilicones or perfluorinated oils;

higher fatty acids, such as myristic acid, palmitic acid, stearic acid, behenic acid, oleic acid, linoleic acid, linolenic acid or isostearic acid;

higher fatty alcohols, such as ketanol, stearyl alcohol or oleyl alcohol;

poly(methylfluoroalkyl)(dimethyl)siloxanes of formula (I):

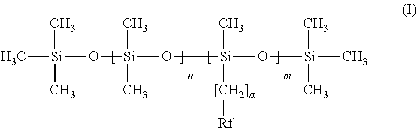

in which:

n represents an integer varying from 5 to 90, in particular from 30 to 80 and especially from 50 to 80,

m represents an integer varying from 1 to 150, in particular from 1 to 80 and especially from 1 to 40,

a represents an integer varying from 0 to 5, and

Rf denotes a perfluoroalkyl radical comprising from 1 to 8 carbon atoms; and

their mixtures.

According to a preferred embodiment, the oil is chosen from fatty esters, such as isopropyl myristate, isopropyl palmitate, butyl stearate, isodecyl stearate, isocetyl stearate, hexyl laurate, isononyl isononanoate, 2-ethylhexyl palmitate, 2-hexyldecyl laurate, 2-octyl-decyl palmitate, 2-octyldodecyl myristate, 2-octyl-dodecyl lactate, di(2-ethylhexyl) succinate, diiso-stearyl malate, glyceryl triisostearate or diglyceryl triisostearate.

The oil of the binder can represent from 10 to 100% by weight, with respect to the total weight of the binder. The binder can be present in the composition according to the invention in a content ranging from 0.5 to 15% by weight, with respect to the total weight of the composition, in particular from 1 to 10% by weight and more preferably from 1.5 to 5% by weight.

The binder may also comprise waxes and pasty fatty substances.

Additives

The composition according to the invention can comprise at lest one other conventional cosmetic ingredient which can be chosen in particular from lipophilic gelling and/or thickening agents, antioxidants, fragrances, preservatives, neutralizing agents, sunscreens, vitamins, moisturizing agents, self-tanning compounds, antiwrinkle active agents, emollients, hydrophilic or lipophilic active principles, agents for combating pollution or free radicals, sequestering agents, film-forming agents, nonelastomeric surfactants, dermo-decontracting active agents, soothing agents, agents which stimulate the synthesis of dermal or epidermal macromolecules and/or which prevent their decomposition, antiglycation agents, agents which combat irritation, desquamating agents, depigmenting, antipigmenting or propigmenting agents, NO synthase inhibitors, agents which stimulate the proliferation of fibroblasts or keratinocytes and/or the differentiation of keratinocytes, agents which act on the microcirculation, agents which act on the energy metabolism of the cells, healing agents, and their mixtures.

In a particular embodiment, the surfactant includes zinc stearate, the binder includes a mineral oil or phenyl trimethicone, and the preservative includes phenoxyethanol.

According to a preferred embodiment, the composition according to the invention can be a cosmetic composition in the liquid or a fluid form comprising:

i) 5 to 50% by weight, preferably 10 to 30% by weight, and more preferably 10 to 20% by weight, with respect to the total weight of the composition, of fusiform particles according to the invention,

ii) 0 to 90% by weight of aqueous phase,

iii) 10 to 80% by weight of fatty phase,

iv) 0 to 20% by weight of pigments.

The amount of aqueous phase in a lotion could be up to 90 wt %, preferably between 60 wt % and 90 wt %, more preferably between 75 wt % and 85wt % of the lotion. The amount of fatty phase in an oil gel could be up to 80 wt %, preferably between 50 wt % and 80 wt %, more preferably between 60 wt % and 75 wt % of the oil gel.

According to a particular embodiment, the composition according to the invention can be an anhydrous composition. The term “anhydrous composition” is understood to mean a composition comprising less than 2% by weight of water, indeed even less than 0.5% by weight of water, and which in particular is devoid of water, the water not being added during the preparation of the composition but corresponding to the residual water contributed by the mixed ingredients.

Aqueous phase

The composition according to the invention may comprise at least one aqueous medium, constituting an aqueous phase, which may form the dispersed phase or the continuous phase of the composition. The aqueous phase may consist exclusively of water.

The water may be a floral water such as cornflower water and/or a mineral water such as eau de Vittel, eau de Lucas or eau de La Roche Posay and/or a spring water.

The aqueous phase may also comprise a mixture of water and of water-miscible organic solvent(s) (miscibility with water of greater than 50% by weight at 25. degree. C.), for instance monoalcohols containing from 1 to 5 carbon atoms such as ethanol and isopropanol, glycols containing from 2 to 8 carbon atoms such as glycerol, propylene glycol, 1,3-butylene glycol, dipropylene glycol or diethylene glycol, glycol ethers such as mono-, di- or tripropylene glycol (C.sub.1-C.sub.4)alkyl ethers and mono-, di- or triethylene glycol (C.sub.1-C.sub.4)alkyl ethers, C.sub.3-C.sub.4 ketones and C.sub.2-C.sub.4 aldehydes, and mixtures thereof.

The aqueous phase (water and optionally the water-miscible organic solvent(s)) may also comprise stabilizers, for instance sodium chloride, magnesium dichloride and magnesium sulfate.

The aqueous phase may also comprise any water-soluble or water-dispersible compound that is compatible with an aqueous phase, such as care active agents, gelling agents, film-forming polymers, thickeners and surfactants, and mixtures thereof.

The compositions according to the invention may comprise at least 22% by weight of water relative to the total weight of the composition. They may for example comprise at least 25% by weight of water relative to the total weight of the composition. The aqueous phase, and for example the water, may be present in the composition according to the invention in a content ranging from 30% to 80% by weight and for example from 35% to 50% by weight relative to the total weight of the composition.

As mentioned previously, the compositions may for example be in a liquid or fluid form, i.e. different from solid forms of stick type, or compacted.

The composition may for example be in the form of an emulsion that may be simple, of the oil-in-water (O/W) or water-in-oil (W/O) type or a multiple emulsion (for example water-oil-water or oil-water-oil) whose use is well known to those skilled in the art. According to one exemplary embodiment of the invention, the composition is in the form of a water-in-oil emulsion.

The composition may also be in the form of a lotion or an oil gel.

It may also comprise a surfactant or a mixture of surfactants whose HLB (hydrophilic/lipophilic balance) is generally adapted to the nature of the emulsion to be stabilized.

As surfactants that may be used in the invention, suitable for obtaining a W/O emulsion, mention may be made of those with an HLB of less than 7 and for example fatty acid esters of polyols, for instance sorbitol or glyceryl mono-, di-, tri- or sesquioleates or stearates, and glyceryl or polyethylene glycol laureates; alkyl or alkoxy dimethicone copolyols whose alkyl or alkoxy chain is pendent or at the end of a silicone backbone, for example containing from 6 to 22 carbon atoms. As surfactants that may be used in the invention for obtaining an O/W emulsion, mention may be made of those with an HLB of greater than 7, for instance fatty acid esters of polyethylene glycol (polyethylene glycol monostearate or monolaurate); polyoxyethylenated fatty acid esters (stearate, oleate) of sorbitol; polyoxyethylenated alkyl (lauryl, cetyl, stearyl, octyl) ethers and dimethicone copolyols. In general, any ionic (cationic or anionic) amphoteric surfactant and any non-ionic surfactant that are well known to those skilled in the art may be used.

The surfactant may be present in the composition in a content ranging from 0.3% to 10% by weight and for example from 1% to 5% by weight relative to the total weight of the composition.

The cosmetic compositions in accordance with the present invention may comprise a fatty phase comprising, for example at least one compound chosen from oils and fatty substances that are solid at room temperature (20-25. degree. C.) and atmospheric pressure, for instance non-micronized waxes and pasty fatty substances, and mixtures thereof.

The fatty phase may be advantageously present in a relative ratio that is sufficient to allow the fusiformed particles under consideration according to the invention to be really dispersed therein.

Thus, the fatty phase may be generally present in a total weight content that is greater than or equal to the total weight content of fusiform particles.

In a preferred embodiment, the liquid composition according to the invention is a skin care product or a make-up product, in particular a foundation, in the form of an aqueous solution, a gel or an emulsion

### BRIEF DESCRIPTION OF THE DRAWINGS

FIG. 1 is a diagram schematically showing the shape of a fusiform particle

FIG. 2 is a diagram showing the temporal change of a volatile ingredient.

FIG. 3 is a diagram showing the amount of the volatile content after 6 hours.

FIG. 4 is a diagram showing the amount of the water content after 6 hours.

### DESCRIPTION OF EMBODIMENTS

Embodiments of the present invention will be described herebelow. It should be noted that the embodiments and descriptions thereof are provided solely to assist in understanding the present invention and should not be construed as limiting the present invention in any way to the embodiments described below.

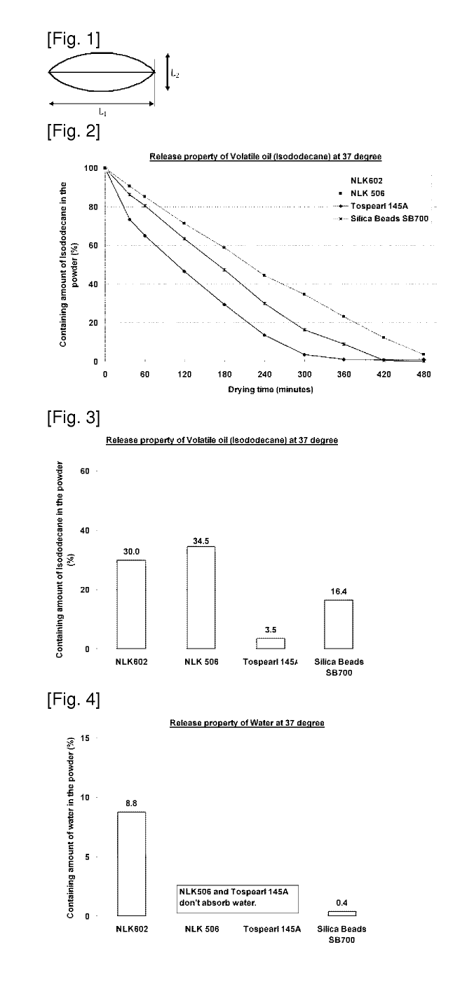

FIG. 1 schematically shows the shape of a fusiform particle. The particle has a rugby ball shape, that is, a major axis along which the diameter is larger than any other diameters, and two minor axes which are perpendicular to the major axis and which are perpendicular to each other. In fact the two minor axes may be defined in any direction as long as the axes are in the plane perpendicular to the major axes. The diameters along major axis L₁ between approximately 0.05 micro meter and approximately 20 micro meter, diameters along minor axes L₂ between approximately 0.03 micro meter and approximately 15 micro meter, and L₁/L₂ between approximately 1.1 and approximately 3.3. As may be understood from FIG. 1, there is a groove on the surface along the major axis, in the particles used in the experiments described below. However, the groove is not necessary.

Table 2 below shows morphology of the particles according to the present invention (Example) which have a fusiform shape, and the comparative particles which have a bowl shape (Reference particle 1 or NLK506), spherical shapes (Reference particle 2 or Tospearl 145A, and Reference particle 3 or Tospearl AQ) or Reference 4 Silica Beads SB700. Their morphology and absorption oil and water are also shown in Table 2.

| | | | | Morphology | | | Absorption Oil and | |
|---|---|---|---|---|---|---|---|---|
| | | Surface | Chemistry | | Mean | | WP Oil | WP Water |
| | Name | treatment | (composition) | Shape | Size (μm) | Size Span | (ml/100 g) | (ml/100 g) |
| Example | NLK602 | NT | Silicone | fusiform | 2.9 | 0.9 | 109.6 | 90.2 |
| | (Takemoto Oil & Fat) | | resin | | | | | |
| Reference 1 | NLK 506 | NT | Silicone | bowl | 2.3 | 0.9 | 155.0 | X |
| | (Takemoto Oil & Fat) | | resin | | | | | |
| Reference 2 | Tospearl 145A | NT | Silicone | sphere | 4.6 | 0.7 | 50.0 | X |
| | (Momentive | | resin | | | | | |
| | Performance Material) | | | | | | | |
| Reference 3 | Silica Beads SB700 | NT | Silica | sphere | 4.6 | 1.3 | 133.0 | 138.0 |
| | (Miyoshi Kasei) | | | | | | | |

NT: no treatment

The protocol of measurement and the definitions are as follows.

Protocol

1. Put 2 g of the powder on glass plate.

2. Add water/oil to the powder a little by little.

3. Knead the powder by spatula until it becomes a paste like form.

Definition

Wet Point (WP): Quantity of solvent necessary to set completely the powder (formation of a paste)

X: Too hydrophobic and wet point could not be measured

Tables 3 and 4 show the composition of water in oil and oil in water dispersions, respectively, to which the particles described in table 1 are to be added.

| INCI NAME | Role | Wt % |
|---|---|---|
| CETYL PEG/PPG-10/1 | WATER-IN-OIL | 1 |
| DIMETHICONE | (W/O) EMULSIFIER | |
| POLYGLYCERYL-4 | SURFACTANT | 0.75 |
| ISOSTEARATE | WATER-IN-OIL (W/O) | |
| | EMULSIFIER | |
| HEXYL LAURATE | SPREADING FACTOR | 0.75 |
| | EMOLLIENT | |
| PEG-10 DIMETHICONE | WATER-IN-OIL (W/O) | 6 |
| | EMULSIFIER | |
| ISOEICOSANE | SOLVENT | 2.5 |
| | HYDROPHOBE | |
| DIMETHICONE | SOLVENT | 2.5 |
| | HYDROPHOBE | |
| ISOSTEARYL NEOPENTANOATE | EMOLLIENT | 0.6 |
| PROPYLPARABEN | PRESERVATIVE | 0.2 |
| CYCLOHEXASILOXANE | SOLVENT | 9 |
| | VOLATILE | |
| | EMOLLIENT | |
| ISODODECANE | SOLVENT | 12 |
| | VOLATILE | |
| | HYDROPHOBE | |
| CYCLOPENTASILOXANE (and) | GELLANT | 14 |
| DISTEARDIMONIUM | THICKENING | |
| HECTORITE (and) ALCOHOL | | |
| DENAT | | |
| WATER | SOLVENT | 35.7 |
| | HUMECTANT | |
| BUTYLENE GLYCOL | SOLVENT | 13 |
| | EMOLLIENT | |
| SODIUM CHLORIDE | CATALYST | 1 |
| METHYLPARABEN | PRESERVATIVE | 0.3 |
| PHENOXYETHANOL | PRESERVATIVE | 0.7 |

| | |
|---|---|
| Total | 100 |

| INCI NAME | Role | Wt % |
|---|---|---|
| WATER | SOLVENT | 47.875 |
| | HUMECTANT | |
| PROPYLENE GLYCOL | PLASTICIZER | 3.75 |
| METHYLPARABEN | PRESERVATIVE | 0.25 |
| GLYCERIN | HUMECTANT | 3.75 |
| | HYDRATING | |
| MAGNESIUM ALUMINUM | ANTI-SEDIMENTARY | 1.25 |
| SILICATE | THICKENING | |
| CELLULOSE GUM | THICKENING | 0.125 |
| TRIETHANOLAMINE | NEUTRALIZING | 1.25 |
| STEARIC ACID | EMULSIFYER | 2.5 |
| GLYCERYL STEARATE | SURFACTANT | 3.125 |
| | EMULSIFYER | |
| | THICKENING | |
| GLYCERYL ISOSTEARATE | SURFACTANT | 3.75 |
| | EMULSIFIER | |
| PHENYL TRIMETHICONE | SOLVENT | 3.75 |
| ISONONYL ISONONANOATE | EMOLLIENT | 8.75 |
| | SPREADING FACTOR | |
| BUTYLPARABEN | PRESERVATIVE | 0.125 |
| CYCLOPENTASILOXANE | SOLVENT | 17.5 |
| | VOLATILE | |
| | SPREADING FACTOR | |
| PROPYLPARABEN | PRESERVATIVE | 0.375 |
| SODIUM LAUROYL | SURFACTANT | 1.875 |
| SARCOSINATE | | |

| | |
|---|---|
| Total | 100 |

Each one of the particles were added to the water in oil dispersion (RAJ1/4) and the oil in water dispersion (RAJ1/6), the liquid was spread on a dark (black) sheet of paper at the thickness of 50 micro meter, laid at the temperature of 37 degrees Celcius for 6 hours so that the dispersion dries, and the reflectivity of the layer was measured. Since the sheet of paper on which the liquids are applied is black, the reflection is 23.6 (very low) if the layer resultant from the liquid is transparent. In contrast, the reflection is high if the layer resultant from the liquid is white or powdery. Table 4 below shows the thus measured refractive indices. Refractive indices of Example 1 (the water in oil dispersion containing the fusiform particles according to the present invention), Example 2 (the oil in water dispersion containing the fusiform particles according to the present invention) are very low, so do the refractive indices of Reference 2A and 2B (the water in oil and the oil in water dispersions containing Tospearl). In contrast, References 1A, 4A, 1B and 4B depict high reflective indices when the liquid is dried 6 hours after application indicating that the surface to which the liquids are applied looks dry and powdery and therefore not enough esthetic.

The protocol and instruments used for the transparency measurement were as follows:

Protocol

1. Apply cosmetic emulsion containing the filler on Contrast card to form a 50 micro meter thick layer.

2. Measure a color of the film on the black side of Contrast card with a colorimeter after drying it at 37 degrees Celsius for 6 hours.

Instruments

Colorimeter: Konica Minolta CR-400

Contrast card: Erichsen, TYP 24/5

TABLE 5
Refractive Indices

| Filler content | | 0% | 10% | 20% | 30% |
|---|---|---|---|---|---|
| Water in Oil | Base only | 26.6 | | | |
| (RAJ1/4) | Example A | | 26.4 | 26.3 | 26.2 |
| | Example 1A | | 43.6 | 62.0 | 77.2 |
| | Example 2A | | 26.3 | 26.5 | 26.6 |
| | Example 3A | | 26.6 | 47.1 | 65.2 |
| Oil in Water | Base only | 23.4 | | | |
| (RAJ1/6) | Example B | | 25.5 | 26.5 | 27.6 |
| | Example 1B | | 28.0 | 57.1 | 75.5 |
| | Example 2B | | 23.3 | 23.6 | 24.0 |
| | Example 3B | | 26.7 | 39.6 | 59.0 |

FIG. 2 shows the evolution in time of the amount of oil contained in the above-described particles. The horizontal axis designates the time in minutes passed from the beginning of the test and the vertical axis shows the amount of a volatile oil (isododecane) contained in the particles. At t=0, the particles contained the amount that can be contained at saturation in the particles. As may be understood from the figure, Example A and Reference 1A tend to preserve oil content for a longer period of time while the other liquids loses their oil content relatively quickly. Example A and Reference 1A only preserves oil contents after the passage of 420 minutes (7 hours) showing maintaining good preservability of oil contents. This feature indicates the ability to avoid dryness of skin and also slow release of ingredients which may be contained in the fatty phase.

FIG. 3 shows the amount of volatile oil contained in the particles 6 hours after the passage of time. It is clear that Example A and Reference 1A preserve the volatile oil content even during 6 hours while other liquids tend to lose the oil content.

FIG. 4 shows the amount of moisture (water) contained in the particles 6 hours after the passage of time. It is clear that only Example A preserves moisture content even during 6 hours while other liquids tend to lose moisture content.

In view of the results shown by Table 5 and FIG. 3 and FIG. 4 (FIG. 2 also), it should be clearly understood that Examples A and B are the only liquid which depict both transparency and good volatile oil preservation which lead to a good original texture by maintaining transparency as well as the oil and water content during as long as 6 hours or more. Other effects of the present invention must be understood by the specification and the attached drawings.

Examples of formulations are provided as follows.

Lotion

A formulation of a lotion product is described as follows.

| TYPE | INCI NAME | wt % |
|---|---|---|
| ACTIVE COMPOUND | TOCOPHEROL | 0.1 |
| NLK602/Filler (rugby) | METHYLSILANOL/SILICATE | 10.0 |
| | CROSSPOLYMER | |
| SOLVENT | BUTYLENE GLYCOL | 0.8 |
| SOLVENT | ALCOHOL | 7.0 |
| SOLVENT | WATER | 80.0 |
| SOLVENT | GLYCERIN | 1.7 |
| PRESERVATIVE | METHYLPARABEN | 0.2 |
| SURFACTANT | GLYCERETH-25 PCA | 0.2 |
| | ISOSTEARATE | |

| | |
|---|---|
| TOTAL | 100.0 |

A formulation of a milky lotion is described as follows.

| TYPE | INCI NAME | wt % |
|---|---|---|
| ACTIVE COMPOUND | TOCOPHEROL | 0.1 |
| NLK602/Filler (rugby) | METHYLSILANOL/SILICATE | 10.0 |
| | CROSSPOLYMER | |
| POLYMER | XANTHAN GUM | 0.2 |
| SOLVENT | BUTYLENE GLYCOL | 9.0 |
| SOLVENT | WATER | 59.0 |
| SOLVENT | GLYCERIN | 19.0 |
| SOLVENT | PROPYLENE GLYCOL | 2.0 |
| SOLVENT | CAPRYLYL GLYCOL | 0.3 |
| PRESERVATIVE | METHYLPARABEN | 0.2 |
| SURFACTANT | PEG-60 HYDROGENATED CASTOR | 0.2 |
| | OIL | |

| | |
|---|---|
| TOTAL | 100.0 |

A formulation of a water gel is described as follows.

| TYPE | INCI NAME | wt % |
|---|---|---|
| ACTIVE COMPOUND | TOCOPHEROL | 2.5 |
| FATTY COMPOUND | SORBITAN PALMITATE | 0.9 |
| FATTY COMPOUND | ISOPROPYL PALMITATE | 3.0 |
| FATTY COMPOUND | CAPRYLIC/CAPRIC TRIGLYCERIDE | 1.0 |
| NLK602/Filler (rugby) | METHYLSILANOL/SILICATE | 10.0 |
| | CROSSPOLYMER | |
| POLYMER | XANTHAN GUM | 0.5 |
| PRESERVATIVE | PROPYLPARABEN | 0.1 |
| PRESERVATIVE | METHYLPARABEN | 0.3 |
| SILICON | CYCLOPENTASILOXANE | 3.0 |
| SOLVENT | WATER | 68.0 |
| SOLVENT | GLYCERIN | 3.0 |
| SOLVENT | PEG-20 | 1.0 |
| SOLVENT | PROPYLENE GLYCOL | 6.0 |
| SURFACTANT | STEARIC ACID | 0.3 |
| SURFACTANT | GLYCERYL STEARATE (and) | 0.2 |
| | PEG-100 STEARATE | |
| VITAMIN | ASCORBYL GLUCOSIDE | 0.2 |

| | |
|---|---|
| TOTAL | 100.0 |

A formulation of a oily gel is described as follows.

| TYPE | INCI NAME | wt % |
|---|---|---|
| SURFACTANT | SORBITAN STEARATE | 1.5 |
| FATTY COMPOUND | ISOHEXADECANE | 18.0 |
| FATTY COMPOUND | ISONONYL ISONONANOATE | 20.0 |
| POLYMER | HYDROGENATED STYRENE/ | 2.5 |
| | ISOPRENE COPOLYMER | |
| NLK602/Filler (rugby) | METHYLSILANOL/SILICATE | 25.0 |
| | CROSSPOLYMER | |
| SOLVENT | ISODODECANE | 14.0 |
| GELLANT | PETROLEUM DISTILLATES (and) | 19.0 |
| | DISTEARDIMONIUM HECTORITE | |
| | (and) PROPYLENE CARBONATE | |

| | |
|---|---|
| TOTAL | 100.0 |

1. A liquid composition for cosmetic use containing fusiform particles in a cosmetically acceptable medium, the fusiform particles being between approximately 3 wt % and 60 wt % of the liquid foundation and made of an organosilicone having a crosslinked polysiloxane structure, and having diameters along major axis L₁ between approximately 0.05 micro meter and approximately 20 micro meter, diameters along minor axes L₂ between approximately 0.03 micro meter and approximately 15 micro meter, and L₁/L₂ between approximately 1.1 and approximately 3.3.

2. The liquid composition according to claim 1, wherein the fusiform particles are between approximately 5 wt % and 50 wt %, preferably between approximately 10 wt % and approximately 30 wt %, more preferably between approximately 10 wt % and approximately 20 wt % of the liquid foundation.

3. The liquid composition according to claim 1 or 2, wherein the crosslinked polysiloxane comprises first, second and third siloxane units which are SiO₂, R¹ SiO_{1.5} and R²R³SiO, respectively, wherein R¹, R² and R³ are any one of organic groups, same or different, having a carbon atom directly linked to a silicon atom.

4. The liquid composition according to one of claims 1 to 3, wherein molar ratio of the molar sum of the first siloxane unit and the second unit over the third siloxane unit is between 99:1 to 50:50, preferably between 90:10 to 60:40, approximately.

5. The liquid composition according to one of claims 1 to 4, wherein molar ratio of the first siloxane unit with respect to the second siloxane unit is between 23:77 and 40:60, approximately.

6. The liquid composition according to any one of claims 3 to 5, wherein R¹ and at least one of R² and R³ are selected from the group consisting of epoxy group, (meta) acryloxy group, mercaptoalkyl group, aminoalkyl group and organic groups having any one of preceding groups.

7. The liquid composition according to one of claims 1 to 6, wherein the fusiform particles have slits along their major axes.

8. The liquid composition according to one of claims 1 to 7, which further comprises at least one compound chosen from water, hydrophilic solvents, lipophilic solvents, oils, waxes and pasty fatty substances, and mixtures thereof.

9. The liquid composition according to claim 8, wherein the oils are chosen from volatile or non-volatile hydrocarbon-based oils of animal, plant or mineral origin, synthetic oils, silicone oils and fluoro oils, and mixtures thereof.

10. The liquid composition according to one of claims 1 to 9 which is in the form of an aqueous solution, a lotion, an oil gel, an oil-in-water or water-in-oil simple emulsion or a multiple emulsion.

11. The liquid composition according to one of claims 1 to 10 which further comprises one of a filler, a pigment and another cosmetically acceptable ingredient.

12. The liquid composition according to claim 11, wherein:
the filler is selected from the group consisting of talc, mica, silica, kaolin, powder formed of polyamide, of poly-beta-alanine and of polyethylene, powders formed of tetrafluoroethylene polymers, lauryllysine, starch, boron nitride, polymeric hollow microspheres of poly(vinylidene chloride)/acrylonitrile or of acrylic acid copolymers, silicone resin microbeads, particles formed of poly-organosiloxane elastomers, precipitated calcium carbonate, magnesium carbonate, basic magnesium carbonate, hydroxyapatite, barium sulphate, aluminium oxides, polyurethane powders, composite fillers, hollow silica microspheres, glass or ceramic microcapsules, or metal soaps derived from organic carboxylic acids having from 8 to 22 carbon atoms, preferably from 12 to 18 carbon atoms, for example zinc stearate, magnesium stearate, lithium stearate, zinc laurate and magnesium myristate;
the pigment is selected from the group consisting of titanium dioxide, optionally surface treated, zirconium or cerium oxides, and also zinc, iron (black, yellow or red) or chromium oxides, manganese violet, ultramarine blue, chromium hydrate and ferric blue, and metal powders, such as aluminium powder or copper powder, and their mixtures; and
the another cosmetically acceptable ingredient is selected from the group consisting of waxes, preservatives, cosmetic active principles, moisturizing agents, UV screening agents, thickeners, water, surfactants, binders and fragrances.

13. The liquid composition according to claim 12, wherein the surfactant includes zinc stearate, the binder includes a mineral oil or phenyl trimethicone, and the preservative includes phenoxyethanol.

14. The liquid composition according to any one of claims 1 to 13 wherein the liquid composition is a skin care product or a make-up product, in particular a foundation, in the form of an aqueous solution, a gel or an emulsion.
